(19) United States
(12) Patent Application Publication
(10) Pub. No.: US 2010/0292696 A1
(43) Pub. Date: Nov. 18, 2010

(54) Device for Securing to a Support, Assembling Piece for this Device, and Method for Securing a Device to a Support

(76) Inventors: Christophe Chantelot, Lille (FR); Victor Katz, Levallois (FR); Bertrand Xavier François Gauneau, Lyon (FR)

Correspondence Address: COOPER & DUNHAM, LLP, 30 Rockefeller Plaza, 20th Floor, NEW YORK, NY, 10112, US

(21) Appl. No.: 11/921,841

(22) PCT Filed: Jun. 6, 2006
(86) PCT No.: PCT/FR2006/001265

§ 371 Date: Jun. 25, 2010

(30) Foreign Application Priority Data
Jun. 6, 2005 (FR) 0505746

(51) Int. Cl. A61B 17/80; F16B 43/00

(52) U.S. Cl. 606/70; 411/533; 606/280

(57) ABSTRACT

The invention relates to a device (1) comprising an assembling piece (3) provided with a hole (4), and a fixing element (5) having a shank (6) as well as head (7). The device (1) has a locking screw (8a) of the head (7), characterized in that the hole (4) is shaped for producing a stop (9) for the head and for enabling the head (7), when it rests against the stop (9), to be displaced inside the hole (4) in the plane of the cross-section of the hole (4), the hole (4) having an inside thread in which the locking screw (8A) is to be screwed so that the locking screw (8A) rests against the head (7) and thus presses the latter against the stop (9). The invention also relates to fixing devices, in particular, surgical implants.

### TECHNICAL FIELD

The present invention relates to the technical field of devices designed to be secured to a support, e.g. for the purpose of consolidating or repairing said support, or for connecting the support to some other body, or indeed for providing the support with an additional element for functional and/or decorative purposes.

The present invention relates in particular to the general technical field of surgical fastener implants such as plates that are designed to promote osteosynthesis of bone fragments, and in particular to the field of epiphyseal plates or shaft plates.

The present invention relates to a device for securing to a support, said device comprising firstly an assembly part provided with at least one hole defining a hole section, and secondly a fastener member for fastening the assembly part to the support, said fastener member comprising a shank designed to be anchored in the support and a head surmounting said shank and designed to be received within the hole, said device including at least one lock screw for locking the head to secure the head to the assembly part.

The present invention also relates to a method of securing a device to a support, said device comprising firstly an assembly part provided with at least one hole defining a hole section, and secondly a fastener member for fastening the assembly part to the support, said fastener member comprising a shank together with a head surmounting said shank and designed to be received within the hole, said method comprising a step of positioning the head in the hole, an anchoring step during which the shank is anchored in the support, and a step of securing the head with the assembly part with the help of at least one lock screw.

The invention is directed more particularly to a surgical device of the implant kind for assembly to a bone or to bone fragments, however it can also be applied in any other field, such as building or do-it-yourself (DIY) for example.

The present invention also relates to an assembly part as such for implementation in a device as specified above.

Below, for purely illustrative and non-limiting purposes, reference is made more generally to the medical field, and more particularly to the bone surgery sector which is the preferred field of application of the invention.

### PRIOR ART

Surgical osteosynthesis plates are known in the prior art that are designed to be fastened on the ends of bones in order to reinforce them, or to repair them if they have been fractured, for example.

Such plates, including epiphyseal plates and shaft plates, can be implemented on numerous types of bone, for example on the epiphysis of the radius in the event of a wrist fracture, or the epiphysis of the humerus in the event of a fracture of the head of the humerus.

Those known plates are generally constituted by a substantially plane and rectilinear metal strip that flares at one of its ends so as to form a spatula. Fastener holes are formed in regularly spaced-apart manner over the entire plate, i.e. both in the strip portion and in the spatula portion.

The plate is designed to be pressed against the bone for reinforcement or repair, so as to position the spatula towards the end of the bone for reinforcing or repairing, with the strip extending towards its opposite end. The plate is secured to the bone by screw-fastening into the bone mass, using screws that are engaged through the holes in the plate.

An epiphyseal plate for the radius can thus be substantially T-shaped, the rectilinear strip forming the upright of the T-shape, while the spatula forms the cross-bar of the T-shape. Other shapes naturally also exist in the prior art. For example, a plate designed to reduce a fracture of the head of the humerus may be substantially Y-shaped with the rectilinear strip forming the upright of the Y-shape while the spatula forms the two arms of the Y-shape.

It is also known to provide osteosynthesis plates with fasteners holes that are oblong in shape, so as to give the surgeon a certain amount of scope for adjustment in translation before fully tightening the corresponding screws. Such an oblong hole is generally positioned in the rectilinear strip.

Such a facility for performing adjustment is found to be very useful in practice, given the fact that a bone, and in particular a fractured bone, is a moving biological support on which the plate needs to be fastened in secure manner so as to fulfill its therapeutic function.

Nevertheless, although conventional plates enable the position of the plate against the bone to be adjusted properly, by virtue of having the above-mentioned oblong hole, they nevertheless present serious drawbacks.

Firstly, the extent to which the bone and the plate are secured together depends on the compression force obtained by the screw-fastening pressing the plate against the bone. Given the different hardnesses of the plate and of the bone (where bone material is generally softer than the metal material from which the plate is made), it can happen that the bone is damaged at the interface between the plate and the bone. This damage can lead to a certain amount of slack between the plate and the bone, which slack necessarily leads to clearance between the head of the screw and the plate.

Such clearance is manifestly harmful to the therapeutic effectiveness of the plate since it can lead to a certain amount of freedom in movement being reestablished between the fragments of bone that ought on the contrary to be held securely relative to one another. Under extreme circumstances, such clearance can lead to the plate being torn off, with all the harmful or even dramatic consequences that can have for the patient.

This problem can be encountered at each of the screws used for fastening the plate to the bone.

This problem arises in particular when the hole is oblong, since such a hole holds the head of the screw less securely than does a conventional circular hole. The additional degree of freedom in sideways translation that the oblong hole affords to the head of the screw thus accentuates the risk of slack appearing between the screw and the plate in the oblong hole, which risk can lead to the above-mentioned harmful consequences.

Document EP-1 221 308 describes a plate provided with two holes made by machining two housings that merely intersect. Each of those two housings presents a spherical bearing surface against which the head of a bone screw comes to bear, said head presenting a spherical shape complementary to the shape of the bearing surface. The head of the screw is thus captured in one or other of the two spherical bearing surfaces and it cannot slide from one bearing surface to the other while continuing to bear thereagainst. The system described in that document therefore does not enable the surgeon to perform fine adjustment in translation of the plate on the bone, after the bone screw has been subjected to pre-tightening for positioning purposes.

Document US-2004/0127899 is content merely to describe a system implementing firstly a plate pierced by an oblong hole provided at its perimeter with a groove, and secondly a bone screw having its head surrounded by a resilient ring. The head of the screw surrounded by its ring is forced into the orifice so that the ring is housed in the groove. The head of the bone screw is also pierced axially by an internally threaded hole into which a threaded cap is screwed in order to prevent the screw from moving. The forced insertion of the screw into the orifice can nevertheless be difficult to perform in a manner that is entirely reliable during an operation, and it leads to an increased risk of the bone support being damaged, or separating from the resilient ring. Furthermore, tightening the threaded cap leads to a force being exerted for preventing movement on the resilient ring only, such that clearance can remain between the ring and the head of the screw, with there also being the risk of the ring escaping if excessive screw-tightening force is applied to the cap.

Thus, at present, there exists no device that makes it possible to benefit from the advantages of an oblong hole in terms of ease and accuracy of positioning, while also benefiting from excellent mechanical stability, and minimizing the risk of any slack or clearance appearing in the assembly between the plate, the screw, and the bone.

### SUMMARY OF THE INVENTION

Consequently, an object of the invention is to provide a remedy to the various drawbacks specified above and to propose a novel device, in particular a surgical device, designed to be secured to a support, in particular a bony support, and of a design that makes it possible to achieve an assembly between the device and the support that is particularly stable from a mechanical point of view, while facilitating adjustment of the position of the device relative to the support.

Another object of the invention seeks to provide a novel device, in particular a surgical device, designed to be secured to a support, in particular a bony support, that enables the device to be guided relative to the support while the device is being secured to the support.

Another object of the invention seeks to provide a novel device, in particular a surgical device, designed to be secured to a support, in particular a bony support, that makes it possible to achieve a device-and-support assembly that is particularly stable, even when the device is not fully in contact with the support.

Another object of the invention seeks to provide a novel device, in particular a surgical device, designed to be secured to a support, in particular a bony support, that enables the device and the support to be assembled together in particularly rigid manner.

Another object of the invention seeks to provide a novel device, in particular a surgical device, designed to be secured to a support, in particular a bony support, that can be dismantled.

Another object of the invention seeks to provide a novel device, in particular a surgical device, designed to be secured to a support, in particular a bony support, that can be implemented using a standard fastener member.

Another object of the invention seeks to provide a novel device, in particular a surgical device, designed to be secured to a support, in particular a bony support, that enables the device and the support to be assembled together quickly and reliably in a manner that is stable from a mechanical point of view.

Another object of the invention seeks to provide a novel device, in particular a surgical device, designed to be secured to a support, in particular a bony support, that is of a structure that is particularly simple and inexpensive, and that can be used by performing techniques that are very close to present techniques.

Another object of the invention is to provide a novel method of securing a device, in particular a surgical device, to a support, in particular a bony support, that is particularly simple and fast to implement, while also making it possible to obtain a device-and-support assembly that is particularly firm, accurate, and stable.

Another object of the invention is to provide a novel method of fastening a device, in particular a surgical device, to a support, in particular a bony support, in which the sequence of operations is very close to the sequence of operations performed in a conventional securing method.

The objects given to the invention are achieved with the help of a device for securing to a support, said device comprising firstly an assembly part provided with at least one hole defining a hole section, and secondly a fastener member for fastening the assembly part to the support, said fastener member comprising a shank designed to be anchored in the support and a head surmounting said shank and designed to be received within the hole, said device including at least one lock screw for locking the head to secure the head to the assembly part, the device being characterized in that the hole is shaped to constitute an abutment for the head, said hole also being shaped and dimensioned so that the head, when bearing against the abutment, can be moved within the hole in translation substantially in the plane of the section of the hole, the hole including at least one internal thread into which the lock screw is designed to be screwed so that said lock screw bears against the head and thus presses it against the abutment.

The objects given to the invention are also achieved with the help of an assembly part for a device in accordance with the invention.

The objects given to the invention are also achieved with the help of a method of securing a device to a support, said device comprising firstly an assembly part provided with at least one hole defining a hole section, and secondly a fastener member for fastening the assembly part to the support, said fastener member comprising a shank together with a head surmounting said shank and designed to be received within the hole, said method comprising a step of positioning the head in the hole, an anchoring step during which the shank is anchored in the support, and a step of securing the head with the assembly part with the help of at least one lock screw, the method being characterized in that the hole is shaped to form an abutment for the head, said hole also being shaped and dimensioned so that the head, when bearing against the abutment, can be moved within the hole in translation substantially in the plane of the section of the hole, the hole including at least one internal thread in which the lock screw is designed to be screwed, said step of securing the head with the assembly part comprising screwing the lock screw into said internal thread so that said lock screw bears against the head and thus presses it against the abutment.

### BRIEF DESCRIPTION OF THE DRAWINGS

Other advantages and objects of the invention appear in greater detail on reading the following description and with the help of the accompanying drawings provided purely by way of non-limiting illustration, and in which:

FIG. 1 is a perspective view from above of a first embodiment of a device in accordance with the invention for a surgical application, and in particular for radial epiphyseal osteosynthesis of the radius;

FIG. 2 is a perspective view from below showing the FIG. 1 device;

FIG. 3 is a perspective view showing an embodiment detail of the FIG. 1 device, in which the means for locking the proximal portion are not shown;

FIG. 4 is a perspective view showing the FIG. 3 embodiment detail with the locking member in a first configuration;

FIG. 5 is a side view in section showing the embodiment detail of FIGS. 3 and 4, in which the locking member is in a second configuration;

FIG. 6 is a perspective view showing the embodiment detail of FIGS. 3 to 5 in which locking means are implemented comprising two elements;

FIG. 7 is a perspective view from above showing an assembly part forming a portion of a device constituting a second embodiment of the invention;

FIG. 8 is a section view showing a third embodiment of a device in accordance with the invention;

FIG. 9 is a fragmentary perspective view from above showing an assembly part forming a portion of a device forming a third embodiment of the invention;

FIG. 10 is a fragmentary perspective view from the side showing the FIG. 9 device; and

FIG. 11 is a fragmentary section view showing the FIG. 10 device.

### BEST MANNER OF PERFORMING THE INVENTION

The invention relates to a device 1 for securing to a support 2 (shown in FIGS. 5 and 8) in order to form an assembly, i.e. a unit together with the support 2. In other words, the device 1 is designed to be coupled to the support 2 in such a manner as to form a rigid part.

Below, reference is made more particularly to a device 1 that is preferably of a surgical nature, with the support 2 being preferably constituted by bone material.

Nevertheless, in the meaning of the invention, the device 1 could be of any kind and could be used in fields that are far removed from the medical sector, such as, for example, the fields of: building, woodworking, or DIY. In this respect, the invention is not limited to a particular type of support 2, the support may, be of any kind, and in particular it may for example be made of wood, concrete, cement, or synthetic material.

In a preferred embodiment, shown in the figures, the device 1 is for performing osteosynthesis at the ends of bones, or in other words epiphyseal osteosyntheses, regardless of the type of bone under consideration.

The device 1 of the invention comprises an assembly part 3 for applying against and fastening to the support 2.

In the preferred application of the invention to surgery, the assembly part 3 is advantageously a surgical instrument or implant designed to be associated with a support 2 in the form of bone.

Preferably, the assembly part 3 is a surgical osteosynthesis plate, e.g. for reducing a bone fracture.

Under such circumstances, the plate forming the assembly part 3 is designed to provide a mechanical connection between the various fragments of the fractured bone, each fragment being connected to the plate 3 in a manner that is described in greater detail below. In a preferred variant of the invention, corresponding to the embodiment shown in the figures, the assembly part 3 comprises a plate for osteosynthesis at the end of bones, and more particularly comprises an epiphyseal plate or a shaft plate.

By way of example, the assembly part 3 may comprise a proximal plate for the humerus, a (medial or lateral) splint plate for the humerus, a proximal plate for the radius (in the event of a fracture to the head of the radius, for example), or a collar bone plate. When the device 1 is more specifically intended for the lower limbs, the assembly part 3 may comprise, for example, a supercondyl plate for the femur, a plate for a proximal fracture of the femur, a medial or lateral proximal plate for the tibia, or indeed a plate for a fracture of the lower extremity of the tibia.

In the example shown specifically in the figures, the assembly part 3 is an anterior plate for the distal radius. As is well known in the field, the surgical plate forming the assembly part 3 is preferably made of a metal presenting the usual properties required for use in the medico-surgical field.

As is also well known per se, said plate presents a T-shape. The upright of the T-shape is constituted by a substantially plane and rectilinear metal strip 3A while the cross-bar of the T-shape is constituted by a spatula 3B, likewise presenting a shape that is substantially plane and rectilinear. The strip 3A and the spatula 3B form a single part, being united via a progressive flare at one of the ends of the strip 3A. The spatula 3B is preferably slightly curved relative to the strip 3A so that the plate as a whole fits better to the anatomy of the bone that it is to reinforce or repair.

As is also well known in the field, the spatula 3B is for being positioned close to the end of the bone (epiphysis), in the vicinity of the joint.

As for the strip 3A, it is designed to extend in the long direction of the bone towards its other end, in the vicinity of the shaft or on the shaft.

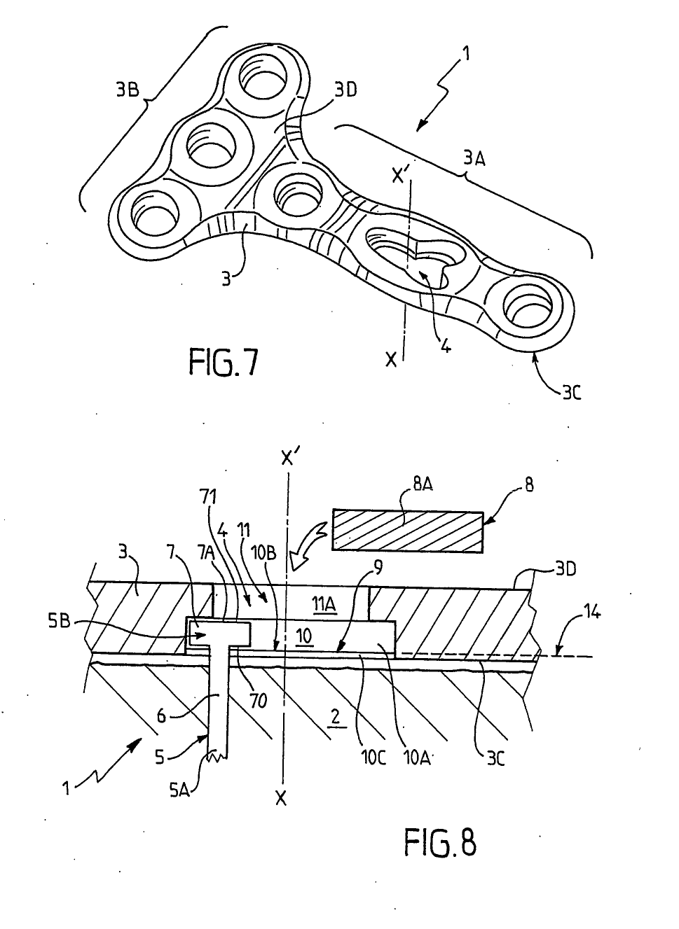

In accordance with the invention, the assembly part 3 is provided with at least one hole 4. The hole 4 is preferably a through hole, formed right through the thickness of the assembly part 3, between the internal face 3C of the assembly part 3 that is to face the support 2, and the opposite external face 3D of the assembly part 3. The hole 4 may be made by removing material from the thickness of the assembly part 3, e.g. using conventional machining methods. The hole 4 extends generally between the internal and external faces 3C and 3D in an axial direction X-X′ (shown for example in FIGS. 7 and 8), which direction corresponds to the depth of the hole and, in the example shown in the figures, is substantially perpendicular to the plane occupied by the strip 3A.

The hole 4 also defines a hole section extending between the walls defining the hole in a plane substantially perpendicular to the axial direction X-X′. The section of the hole 4 preferably occupies a plane that is substantially coplanar with the planes occupied by the internal and external faces 3C and 3D. In other words, the section of the hole 4 corresponds to the length and to the width of the hole 4, while the axial direction X-X′ corresponds to the depth of said hole.

The device 1 in accordance with the invention also comprises a fastener member 5 for fastening the assembly part 3 to the support 2. The fastener member 5 presents a distal portion 5A for anchoring in the support 2, and for example penetrating therein, together with a proximal portion 5B designed to be received, i.e. housed, within the hole 4.

As shown in the figures, the fastener member 5 preferably comprises a shank 6 forming the distal portion 5A and a head 7 surmounting and extending said shank 6 and forming the proximal portion 5B. As is well known to the person skilled in the art, the head 7 is preferably provided with a socket 7A, e.g. a hexagonal socket, designed to receive a corresponding driver key (not shown) in order to anchor the shank 6 in the body 2 by screw-fastening.

In conventional manner, the head 7 presents a diameter (or at least a radial extent) that is greater than that of the shank 6, and may in particular present a collar, e.g. of substantially frustoconical shape (cf. FIG. 5). Preferably, the head 7 presents firstly an inside face 70 from which the shank 6 extends, and secondly an opposite, outside face 71, within which the socket 7A is formed, for example.

Naturally, it is entirely possible, within the ambit of the invention, to envisage making use of a fastener member 5 having a head 7 that does not form the proximal portion 5B that is to be received within the hole 4. For example, the head could be situated beyond the proximal portion 5B in question, which is then interposed along the length of the fastener member 5 between the head 7 and the distal portion 5A. Under such circumstances, the head 7 may project from the hole 4 (variant not shown).

Preferably, the shank 6 is threaded. The fastener member 5 then preferably constitutes a screw designed to be screwed into the support 2. It is naturally possible, without thereby going beyond the ambit of the invention, to envisage the fastener member 5 being constituted by any alternative fastener means well known to the person skilled in the art, for example by a substantially smooth shank without a thread, such as a nail or a pin.

As shown in the figures, the hole 4 is advantageously shaped firstly to allow the shank 6 to pass therethrough, and secondly to constitute an abutment 9 for the head 7. The section of the hole 4 is thus sufficiently great relative to the diameter of the shank 6 to enable said shank 6 to be threaded through the hole 4 in the axial direction X-X′, until the head 7 is stopped in its axial displacement from the external face 3D to the internal face 3C by a stop means, constituted by a specific conformation of the hole 4, forming an abutment 9.

Naturally, the head 7 is shaped to co-operate with the abutment 9, i.e., as shown in the figures, it presents in particular a diameter that is greater than the diameter of the shank 6, so as to be capable of bearing against the abutment 9.

In preferred manner, the abutment 9 thus forms a bearing surface for the head 7, which bearing surface extends substantially in a bearing plane 14.

In accordance with the invention, the hole 4 is shaped and dimensioned so that the head 7 of the fastener member 5, when it bears against the abutment 9, is capable of being moved within the hole 4 in translation substantially in the plane of the section of the hole 4. Thus, the head 7 can be moved in translation sideways within the hole 4 while continuing to remain pressed against the abutment 9, i.e. the head 7 can slide over the abutment 9 in the plane of the section of the hole. This technical characteristic enables the surgeon to perform fine pre-positioning of the plate 3, by tightening the screw forming the fastener member 5 in the bone 2 so that the head 7 comes to bear against the abutment 7, with the screw tightening still allowing the head 7 to slide sideways over the abutment 9; the surgeon can then cause the plate 3 to slide, where necessary, prior to final tightening of the fastener member 5.

In other words, the proximal portion 5B formed by the head 7 can move parallel to a plane that advantageously extends substantially perpendicularly to the depth axial direction X-X′ of the hole 4. This means that the relative shapes and dimensions of the hole 4 and of the proximal portion 5B are selected specifically relative to each other to enable the proximal portion 5B preferably formed by the head 7 to move “sideways” within the hole 4, in translation inscribed in a plane that is substantially perpendicular to the direction X-X′.

According to the invention, the hole 4 and the proximal portion 5B are designed relative to each other to enable the proximal portion 5B, preferably formed by the head 7, to move freely, preferably along a rectilinear path, in the long direction and/or the width direction of the hole 4, parallel to the plane of the section of the hole 4. Naturally, that does not exclude the possibility of the proximal portion 5B also being capable of turning within the hole, or indeed being capable of moving in translation along the axial direction X-X′.

The implementation of the technical function of moving sideways in translation as mentioned above depends essentially on co-operation between the head 7 and the hole 4, which need to be designed relative to each other to enable the looked-for adjustment in translation to be performed. Consequently, in the meaning of the invention, the hole 4 can present virtually any conformation and dimensions, providing it allows the head 7 to move in translation within the hole 4 parallel to the plane of the section of the hole 4, when the head is resting against the abutment 9.

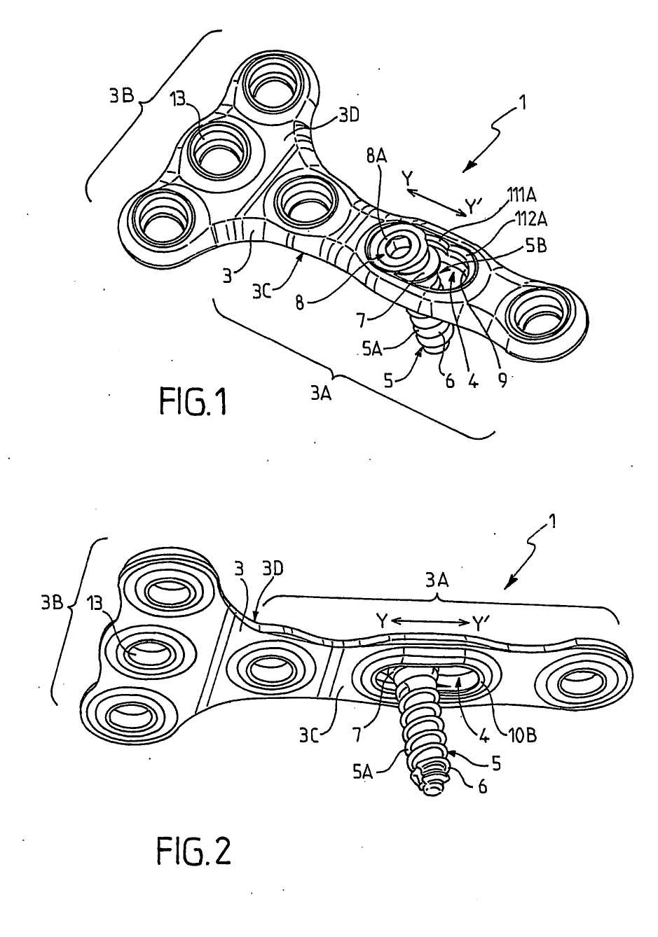

In the examples shown in FIGS. 1 to 8, the plane of the section of the hole 4 along which the head 7 can move is substantially parallel to the internal and external faces 3C and 3D.

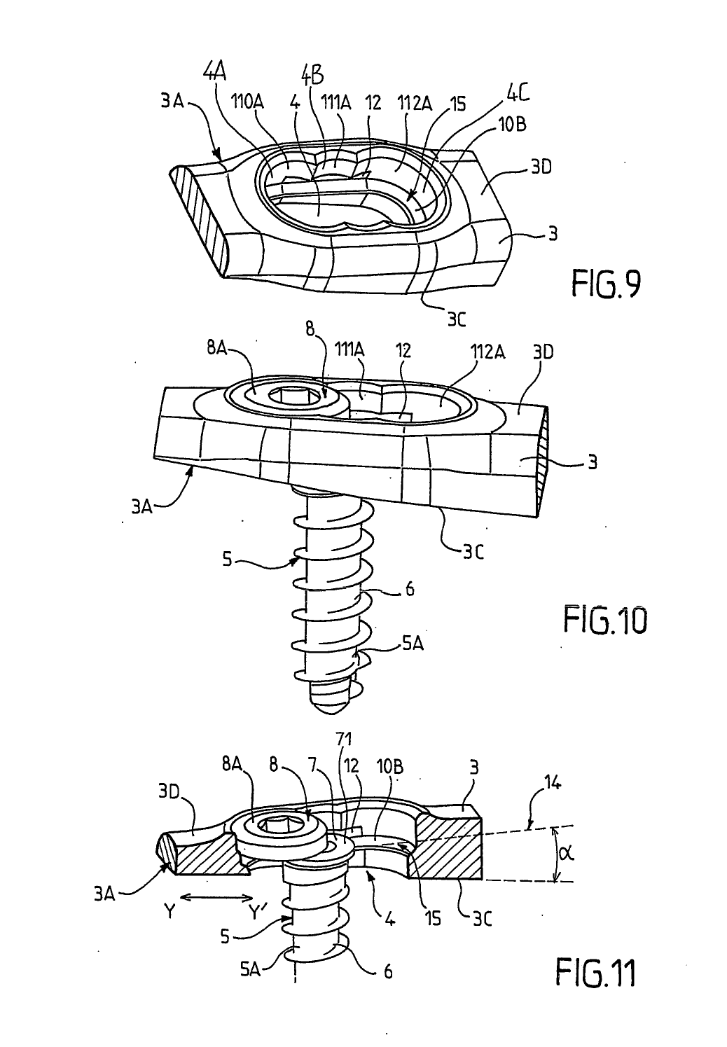

Nevertheless, without thereby going beyond the ambit of the invention, it is possible to envisage that this section plane presents an angle of incidence that is oblique relative to the internal and external faces 3C and 3D, as shown in the variant of FIGS. 9 to 11.

By way of example, and as shown in FIGS. 1 to 8, the hole 4 includes at least one portion of oblong shape, i.e. it is elongate, or indeed essentially longer than it is wide in the plane of the section of the hole 4.

Nevertheless, it is entirely possible to envisage that the hole 4 does not present a portion of oblong shape, e.g. it could present merely a shape that is substantially rounded or polyhedral. In the example of FIG. 7, the hole 4 thus presents substantially a Y-shape, formed by uniting three portions, each of oblong shape.

In a manner that is entirely preferred, the hole 4 nevertheless presents a shape that is oblong, i.e. it is constituted by an oblong hole (as in the variants shown in FIGS. 1 to 6).

Advantageously, and as shown in FIGS. 1 to 6, the oblong hole 4 is formed in the thickness of the rectilinear strip 3A, in the long direction of said strip. As a result, the oblong hole 4 preferably serves to guide the proximal portion 5B in longitudinal translation in the direction Y-Y′ in which the strip 3A extends, i.e. in the long direction of said strip 3A.

Naturally, it is entirely possible to envisage the oblong hole 4 extending transversely relative to the direction Y-Y′ in which the strip 3A extends, without that going beyond the ambit of the invention.

It is also possible to envisage that the plate forming the assembly part 3 has a plurality of oblong holes, e.g. two oblong holes extending perpendicularly relative to each other, a first oblong hole extending in the direction Y-Y′ and being formed in the strip 3A, while the other oblong hole may, for example, be formed in the spatula 3B, in a direction that is substantially perpendicular to the direction Y-Y′, in the direction in which the spatula 3B extends, for example.

The person skilled in the art will naturally be capable, on the basis of the present teaching, to implement any kind of combination of holes, so as to obtain the desired adjustment and/or guidance function(s), without thereby going beyond the ambit of the invention.

In accordance with an important characteristic of the invention, the device 1 includes locking means 8 for locking the proximal portion 5B that is to be received in the hole 4 so as to secure the proximal portion 5B with the assembly part 3. In other words, the locking means 8 is designed to be housed within the hole 4 so as to establish a mechanical connection within the hole 4 between the assembly part 3 and the proximal portion 5B, which mechanical connection serves to prevent substantially any free movement of the proximal portion 5B in the hole 4. By means of this mechanical junction established between the fastener member 5 and the assembly part 3, the mechanical stability of the assembly comprising the part 3 and the support 2 is improved, since even in the event of the contact interface between the part 3 and the support 2 wearing, no harmful or controlled slack is liable to occur between the fastener member 5 and the assembly part 3, given that the proximal portion 5B is secured to the assembly part 3 inside the hole 4.

The invention thus makes it possible to benefit from the advantages of an oblong hole in terms of adjustment, positioning, and guidance, without suffering from the drawbacks in terms of the mechanical instability that are most particularly inherent to such an oblong hole, particularly in the event of the surface of the support 2 suffering wear.

Advantageously, the locking means 8 are designed to secure the proximal portion 5B to the assembly part 3 independently of the distal portion 5A being anchored in the support 2. In other words, the locking means 8 are designed to act completely independently in the hole 4 on the proximal portion 5B, regardless of the fastening force engaging the assembly part 3 and the support 2 as exerted by the fastener member 5, and in particular when the fastener member 5 comprises a threaded shank 6, regardless of the screw-tightening force exerted on said threaded shank 6 via the socket 7A.

Thus, even if the part 3 is not firmly clamped by the fastener member 5 against the support 2, the locking means 8 is designed to exert its mechanical connection action completely independently within the hole 4 between the part 3 and the proximal portion 5B, as preferably constituted by the head 7 in the example shown in the figures. This technical characteristic serves in particular to make it possible to implement constructions on “piles” or “stilts” in which the surgical plate forming the assembly part 3 is situated, once positioning has been completed, as a distance from the bone support 2, i.e. in which it is not in contact with said bone support 2, as shown for example in FIG. 5. In the example of FIG. 5, the plate forming the assembly part 3 is situated at a significant distance D from the bone 2, but without that harming the overall stiffness of the assembly.

Advantageously, the locking means 8 are designed to clamp the proximal portion 5B and the assembly part 3 together in the hole 4. Under such circumstances, the proximal portion 5B, preferably formed by the head 7, is positively wedged in the hole 4, thus having the effect of eliminating substantially any degree of freedom between the proximal portion 5B and the hole 4, i.e. the proximal portion 5B is held completely stationary relative to said hole 4.

Advantageously, the locking means 8 are releasable, i.e. of a design enabling them to be connected in non-final or reversible manner between the proximal portion 5B and the assembly part 3. More particularly, the locking means 8 are preferably mountable and removable at will by the user.

Naturally, the invention is not limited to such a removable and separable locking means 8, but also covers locking means 8 that are optionally secured permanently to the fastener member 5 and/or to the assembly part 3, and the invention also relates to locking means 8 that enable a permanent and irreversible connection to be established between the proximal portion 5B and the assembly part 3.

Advantageously, the locking means 8 are designed to press the head 7 against the abutment 9 to secure the head 7 forming the proximal portion 5B with the assembly 3.

In this variant, which corresponds to that shown in the figures, the locking means 8 are thus designed to exert a compression force substantially along the axial direction X-X′ against the head 7, which head is interposed between the abutment 9 and the locking means 8, until said head 7 is wedged and held stationary against the abutment 9 by the locking means 8.

For this purpose, the head of the screw 7 preferably presents a substantially plane outside face 71 that is to constitute a force transmission and bearing structure for the locking means 8.

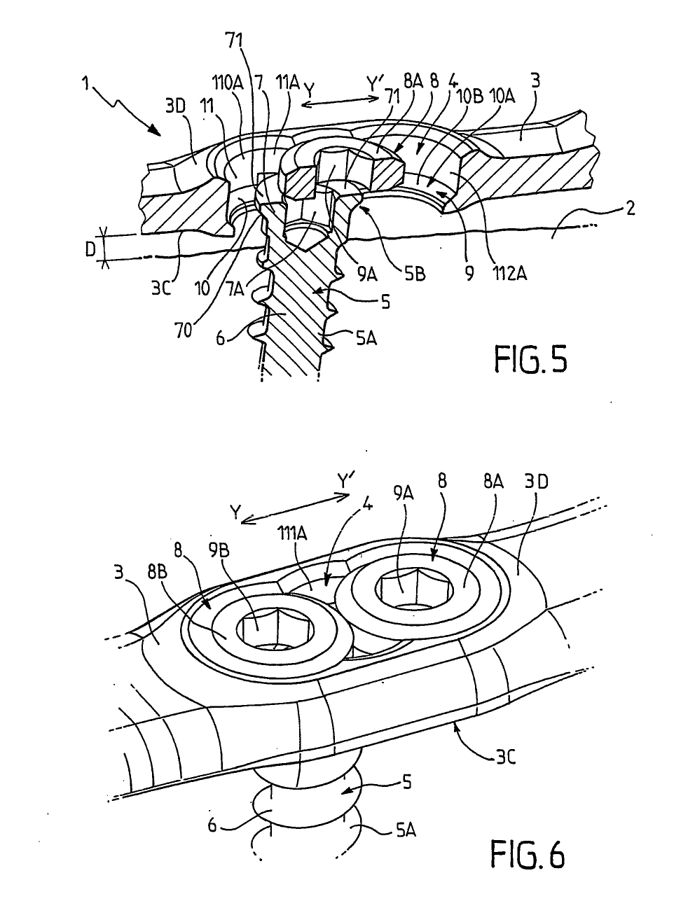

In preferred manner, and as shown in the figures, the locking means 8 comprise at least one lock screw 8A, and in the variant of FIG. 6, they comprise two lock screws 8A and 8B. Thus, the device 1 advantageously includes at least one lock screw 8A, 8B for locking the head 7 so as to secure the head 7 to the assembly part 3. Said heads 8A and 8B may, for example, be in the form of simple threaded washers of substantially cylindrical shape and of uniform diameter.

Said lock screws, that can also be referred to in this application as “check-screws” are preferably provided with corresponding tightening sockets 9A, 9B suitable for co-operating effectively with a suitable driver tool.

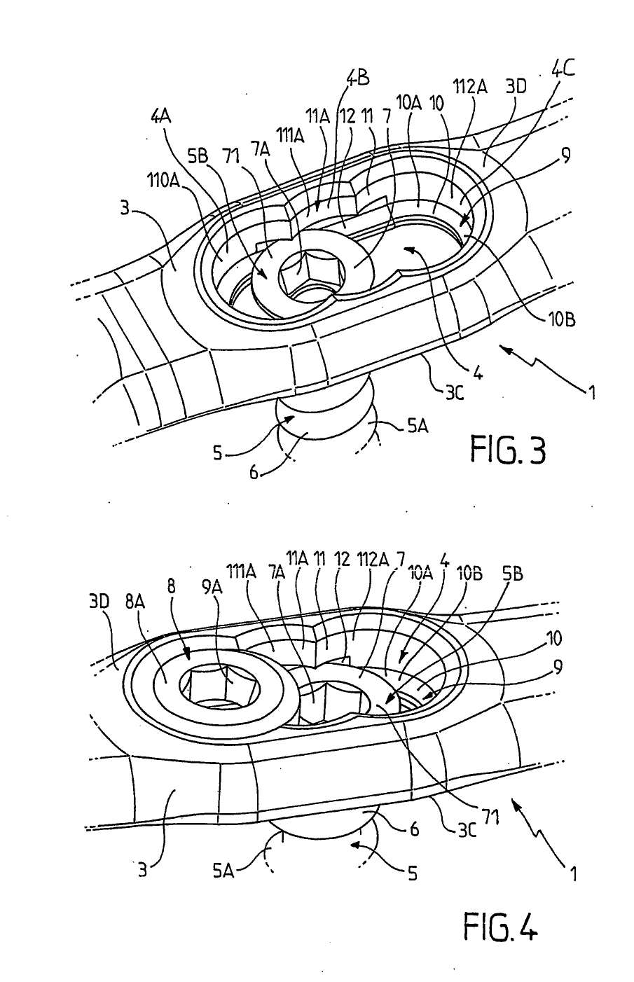

The hole 4 has at least one internal thread in which the lock screw 8A, 8B is designed to be screwed so that said lock screw 8A, 8B bears against the head 7, and thus presses the head against the abutment 9. In the preferred embodiments shown in FIGS. 3 and 9, the hole 4 has three internal threads 4A, 4B, and 4C in which the lock screws 8A, 8B can be screwed.

Thus, by screwing the lock screw(s) 8A, 8B in the hole 4, it is possible to press the head 7 against the abutment 9, thereby mechanically clamping the head 7 relative to the assembly part 3 in the hole 4.

Advantageously, the hole 4 has a bottom stage 10 for facing the support 2. In other words, the bottom stage 10 is preferably situated towards the internal face 3C of the plate forming the assembly part 3. The bottom stage 10 has a bottom orifice 10A presenting a section constriction 10B so as to form the abutment 9.

In the variant shown in FIGS. 1 to 6, the bottom orifice 10A is substantially oblong in shape, with an outline formed by two opposite semicircles interconnected by two parallel rectilinear segments. The section constriction 10B is formed by a shoulder that extends towards the inside of the hole 4, while preferably also being flush with the internal face 3C. Said shoulder thus forms a bottom or seat that is to constitute a bearing surface for the head 7, said bottom or seat being pierced so as to allow the shank 6 to pass through. In other words, the bottom orifice 10B constitutes an oblong bottom spot face that forms the abutment 9 for the head 7, which spot face comprises two parallel rectilinear bearing strips interconnected by semicircular curved strips at their ends.

In the examples shown in FIGS. 1 to 8, the section constriction 10B extends substantially in a bearing plane 14 that is parallel to the internal and external faces 3C and 3D, so as to form a horizontal flat seat for the head 7, said seat extending substantially parallel to the support surface 2.

Nevertheless, it is possible to envisage the section constriction 10B lying in a plane that is oblique within the thickness of the part 3, i.e. on a plane that is inclined relative to the plane in which the part 3 generally extends.

Thus, in the variant of FIGS. 9 to 11, the bearing plane 14 is perceptibly inclined at an angle α relative to the internal face 3C that is to face the support 2, once the part 3 has been put into place on said support 2 in order to be fastened thereto.

The internal face 3C, which is preferably to come directly in contact against the support 2, is itself preferably substantially plane.

Advantageously, the bearing surface extending in the bearing plane 14 is inclined so as to form a ramp for moving the assembly part 3 in translation under the effect of axial thrust exerted by the head 7.

In other words, the axial anchoring force of the fastener member 5 in the support 2 delivers axial thrust on the inclined plane formed by the abutment 9, which axial thrust becomes transformed, because of the wedging effect produced by the relative inclination of the abutment 9 and the plane of the interface between the part 3 and the support 2, into movement in longitudinal translation in the direction Y-Y′ that is substantially perpendicular to the axial direction.

By way of example, the angle of inclination α may be less than 60°, and is preferably less than 45°. Still more preferably, the angle of inclination α lies in the range 1° to 30°.

Because of this ability to cause the part 3 to move in the direction Y-Y′ under the effect of anchoring the fastener member 5 in the support 2, the invention can be used to cause the support 2 to apply compression or traction along said direction Y-Y′.

For this purpose, it suffices to begin by fastening the part 3 in the support 2 at a location that is distinct and remote from the hole 4, e.g. using a screw engaged in the central hole 13 of the spatula 3B. Thereafter, the threaded shank 6 is screwed into the hole 4, after previously positioning said threaded shank 6 towards the zone 15 of the section constriction 10B that is furthest from the internal face 3C. Then, under the effect of the axial thrust of the head 7 against the section constriction 10B forming an inclined plane, a longitudinal force is exerted on the part 3 between the shank 6 and the screw engaged in the hole 13, which force is taken up by the support 2.

Depending on the position of the hole 13 relative to the direction of inclination of the section constriction 10B, the force that is exerted will either be a compression force or a traction force. Naturally, in the preferred example where the device 1 is used surgically for reducing a fracture, it is preferable to implement a compression force so as to optimize mechanical connection of the pieces of bone debris between one another.

In particular in the variant shown in the figures, the bottom orifice 10A constitutes means for guiding in translation the head 7 that forms the proximal portion 5B, said head 7 being capable of sliding in the hole 4 along the section constriction 10B in the longitudinal direction Y-Y′, in the slot defined by the bottom orifice 10A.

Naturally, the bottom orifice 10A may present a shape other than that of an oblong hole, for example it may be substantially circular in shape, as shown in FIG. 8. Under such circumstances, the head 7 can move freely in translation both in the longitudinal direction Y-Y′ and in the transverse direction that is perpendicular thereto.

Advantageously, the hole 4 also has a top stage 11 situated above the bottom stage 10 in the thickness direction X-X′ of the assembly part 3, i.e. that is preferably superposed over said bottom stage 10.

The top stage 11 advantageously has at least one top orifice 11A shaped to co-operate with the locking means 8 so as to make it possible in particular to cause the locking means 8 to be held in the top orifice 11A. Advantageously, the top orifice 11A is internally threaded to enable the lock screw 8A forming the locking means 8 to be screwed therein, said lock screw 8A thus being capable of pressing the head 7 against the section constriction 10B so as to secure the proximal portion 5B formed by the head 7 with the assembly part 3.

Thus, the general concept of the invention lies on the superposition and the co-operation between firstly a bottom stage 10 within which the fastener member 5 can be moved in translation so as to have its position adjusted relative to the plate forming the assembly part 3, and secondly a top stage 11 dedicated to receiving the locking means 8 and to co-operating therewith in order to clamp the head 10 within the hole 4.

The top orifice 11A and the bottom orifice 10A may be superposed substantially exactly (as shown in FIGS. 1 to 6).

It is also possible to envisage that one of the bottom and top orifices 10A, 11A presents a section that is greater than that of the other. For example, and as shown in FIG. 8, the bottom orifice 10A may present a section that is greater than the section of the top orifice 11A, with the shapes and the dimensions of the head 7, of the bottom orifice 10A, of the top orifice 11A, and of the locking means 8 being designed so that, with the help of a screw 8A forming single locking means 8, the head 7 can be locked regardless of the position in which it is to be found within the bottom orifice 10A.

In this embodiment, an example of which is shown in FIG. 8, the bottom orifice 10A presents a shape that is substantially circular and that is provided at its base with an annular inwardly-directed collar 10C that forms the abutment 9. The top orifice 11A is also circular in shape and is situated above the bottom orifice 10A so as to open out therein. The top orifice 11A and the bottom orifice 10A are positioned coaxially. The head 7 of the fastener member 5 is dimensioned so as to be capable of being moved in circular translation movement within the bottom orifice 10A around the collar 10C. The screw 8A forming the locking means 8 is screwed into the top orifice 11A in such a manner as to exert an axial compression force on the head 7 and press it against the inwardly-directed collar 10C. The diameter of the top orifice 11A is greater than that of the bottom orifice 10A, with the ratio of these diameters nevertheless taking account of the diameter of the head 7 so as to enable the screw 8A to be caused to bear against said head 7.

Naturally, it is possible to envisage the diameter of the top orifice 11A and the diameter of the bottom orifice 10A being substantially equivalent. In the embodiment of FIG. 8, it is also possible to envisage the bottom orifice 10A presenting a shape that is not circular, but that is rather oblong, e.g. so as to provide adjusted guidance for the head 7 in translation.

In an alternative embodiment shown in FIGS. 1 to 6, the top stage 11 has a plurality of top orifices 110A, 111A, 112A, all of which are internally threaded. Together the internally threaded top orifices 110A, 111A, 112A (there being three of them in the example shown in the figures), contributes to forming the top orifice 11A.

In the example of FIGS. 1 to 6, the top orifices 110A, 111A, 112A are positioned side by side, preferably substantially in alignment along the longitudinal direction Y-Y′.

Naturally, the internally threaded orifices in question 110A, 111A, 112A need not be in alignment, but could be disposed for example in a triangle, or in any other geometrical pattern.

In the variant shown in FIGS. 1 to 6, the internally threaded top orifices 110A, 111A, 112A are positioned side by side so as to form a resulting single top orifice 11A, while enabling the screws 8A, 8B forming the locking means 8 to be screwed into each of the internally threaded top orifices 110A, 111A, 1112, said screw 8A, 8B thus being capable of pressing the head 7 against the section constriction 10B so as to secure the head 7 to the assembly part 3.

In other words, the circular outlines of the orifices 110A, 111A, 112A interpenetrate so as to form a single orifice 11A, as shown in FIGS. 1 to 6, said interpenetration of the outline nevertheless allowing the screws 8A, 8B forming the locking means 8 to be screwed into any of said orifices, even though the outlines thereof are open, i.e. incomplete, as in the variant of FIGS. 1 to 6.

In the variant in question, it is thus possible to lock the head 7 by screwing a lock screw 8A into one of the orifices 110A, 111A, 112A, e.g. in the central orifice 11A (cf. FIG. 5), or in the left orifice 110A (cf. FIGS. 1, 2, and 4).

It is also possible to envisage securing the head 7 with the assembly part 3 by screwing in two screws 8A and 8B respectively in the left orifice 110A and in the right orifice 112A, the two screws 8A and 8B then becoming substantially tangential in the center of the central orifice 111A (cf. FIG. 6).

Thus, in the embodiment shown in FIGS. 1 to 6, the fastener member 5 is engaged through the resulting hole 11A formed by associating the individual orifices 110A, 111A, 112A, until its head comes to bear in the bottom orifice 10A against the section constriction 10B. The head 7 bearing against the bottom top face that forms the abutment 9 can then slide within the bottom orifice 10A, e.g. in the direction Y-Y′ (this applies for example when the bottom orifice 10A presents an oblong shape extending substantially along said longitudinal direction Y-Y′). Once the position of the head 7 relative to the hole 4 has been determined, the head 7 is locked in the hole 4 by screwing in one or more check-screws 8A, 8B in the individual orifices 110A, 111A, 112A.

Unlike that which is shown in FIGS. 1 to 6, it is naturally possible to envisage the orifices 110A, 111A, 112A being substantially distinct, i.e. having their individual internally threaded outlines substantially closed and separated from one another by boundaries of material. Under such circumstances, where the individual orifices 110A, 111A, 112A do not interpenetrate, the principle of operation nevertheless remains the same, since each of the top individual orifices 110A, 111A, 112A still opens into a single bottom orifice 10A, within the head 7 can be moved in translation, e.g. along the direction Y-Y′ if the bottom orifice 10A is an oblong hole (as shown in the figures).

The variant shown in FIGS. 1 to 6 is nevertheless preferred, since it makes it easy for the user to use the device 1, in particular because it enables the user always to have a good view of the head 7.

Preferably, and as shown in FIGS. 1 to 6, the bottom orifice 10A is oblong in shape, extending in the longitudinal direction Y-Y′, with the top orifice 11A being the result of the internally threaded individual top orifices 110A, 111A, 112A being associated with one another in alignment and directly overlying said bottom orifice 10A. Providing the bottom orifice 10A under the individual top orifices 110A, 111A, 112A also makes it necessary, at least in the variant shown in the figures, to provide a groove 12 under said top orifices 110A, 111A, 112A, so as to provide a uniform section for the bottom orifice 10A, enabling the head 7 to slide in the bottom orifice.

The invention also provides a method of securing a device 1 in accordance with the invention to a support 2, and in particular a method of securing a device 1 in accordance with the invention to a support 2, which method is not a method of surgical or therapeutic treatment.

In particular, the invention relates to a method of securing a device 1 to a support 2 in which said device 1 comprises firstly an assembly part 3 provided with at least one hole 4 defining a hole section, and secondly a fastener member 5 for fastening the assembly part 3 to the support 2, said fastener member 5 comprising a shank 6 together with a head 7 surmounting said shank 6 and designed to be received within the hole 4, the hole 4 being shaped to form an abutment 9 for the head 7, said hole 4 also being shaped and dimensioned so that the head 7, when it comes to bear against the abutment 9, can be moved within the hole 4 in translation substantially in the plane of the section of the hole 4.

In accordance with the invention, the method comprises firstly a positioning step of positioning the head 7 forming the proximal portion 5B in the hole 4, and secondly an anchoring step during which the shank 6 forming the distal portion 5A is anchored in the support 2.

The positioning step and the anchoring step can be performed together, or one before the other.

In accordance with an important characteristic of the method of the invention, said method includes a step of securing the head portion 7 with the assembly part 3 with the help of at least one lock screw 8A, 8B designed to be received in the hole 4, the hole 4 including at least one internal thread 4A, 4B, 4C in which the lock screw 8A, 8B is designed to be screwed, said step of securing the head 7 with the assembly part 3 comprising screwing the lock screw 8A, 8B in said internal thread 4A, 4B, 4C so that said lock screw 8A, 8B bears against the head 7 and thus presses it against the abutment 9.

The method in accordance with the invention advantageously includes a step of moving the proximal portion 5B relative to the hole 4, during which step the proximal portion 5B is moved relative to the hole 4 in translation substantially in the plane of the section of the hole 4.

In this way, the relative position of the part 3 and of the support 2 can be adjusted finely, which is found to be particularly useful when the securing method constitutes a method of securing a surgical device 1, such as an osteosynthesis plate forming the assembly part 3 with a support 2 constituted by bone material.

Advantageously, the movement step is performed after the anchoring step by moving the assembly part 3 relative to the support 2.

There follows a description in greater detail of a securing method in accordance with the invention that makes use of the device 1 shown in FIGS. 1 to 6 for reducing a fracture of the wrist at the distal end of the radius.

Initially, the surgeon positions the plate 3 against the radius 2 so that the spatula 3B is positioned at its epiphysis, as close as possible to the joint surface.

Thereafter, the surgeon fastens a drilling guide, known per se, on the hole 4 and drills a first hole in the bone 2, so as to enable a surgical screw 5 to be screwed in the bone 2 in said first hole.

The surgeon tightens the screw 5 against the abutment 10B in such a manner as to allow the plate 3 to slide against the bone 2, which sliding is guided in the longitudinal direction Y-Y′ by co-operation between the screw 5 and the bottom oblong orifice 10A.

At the end of this pre-positioning step, the surgeon fastens the drilling guide on one of the holes of the spatula 3B, for example on the central hole 13, so as to drill a hole in the epiphysis. Because of the ability of the plate 3 to move in translation along the longitudinal direction Y-Y′, the surgeon can adjust the position of the second hole accurately, taking account in particular of the extent and the location of the fracture, and also the outline of the wrist joint surface.

Thereafter the surgeon screws a second screw into the hole 13.

With these two fastening points, the plate 3 can be accurately adjusted relative to the bone 2. The surgeon can then finish off tightening the screw 5 in the hole 4, e.g. by applying additional tightening force so as to lock the head 7 against the abutment 10B.

When the abutment 10B is inclined (cf. FIGS. 9 to 11), this additional tightening force serves to put the bone 2 under compression between the hole 13 and the hole 4.

Thereafter, the surgeon can seal the screw heads 7 in the holes 4 and 13 with the help of check-screws 8A. In the hole 4, the surgeon can opt to use one or two check-screws 8A, 8B depending on the position of the screw 5 relative to the hole 4, and as shown for example in FIG. 1 and in FIG. 6.

The sealing of the head 7 is found to be particularly appropriate when the device 1 presents an inclined abutment 9 so as to apply compression (cf. FIGS. 9 to 11), since it is then particularly necessary to lock the head 7 firmly in position in the hole 4 so as to avoid any sliding of the head 7 under the effect of resilient return of the bone support 2 acting against the compression force.

The invention thus makes it possible to position the assembly part 3 finely relative to the support 2, and also optionally to apply compression, while also enabling the fastener member 5 to be sealed in said assembly part 3.

### SUSCEPTIBILITY OF INDUSTRIAL APPLICATION

The industrial application of the invention lies in the design, the manufacture, and the implementation of devices that are to be secured to a support, and in particular devices that are designed to implement osteosynthesis of bone fragments.

1. A device (1) for securing to a support (2), said device (1) comprising firstly an assembly part (3) provided with at least one hole (4) defining a hole section, and secondly a fastener member (5) for fastening the assembly part (3) to the support (2), said fastener member (5) comprising a shank (6) designed to be anchored in the support (2) and a head (7) surmounting said shank (6) and designed to be received within the hole (4), said device (1) including at least one lock screw (8A, 8B) for locking the head (7) to secure the head (7) to the assembly part (3), the device being characterized in that the hole (4) is shaped to constitute an abutment (9) for the head (7), said hole (4) also being shaped and dimensioned so that the head (7), when bearing against the abutment (9), can be moved within the hole (4) in translation substantially in the plane of the section of the hole (4), the hole (4) including at least one internal thread (4A, 4B, 4C) into which the lock screw (8A, 8B) is designed to be screwed so that said lock screw (8A, 8B) bears against the head (7) and thus presses it against the abutment (9).

2. A device (1) according to claim 1, characterized in that the hole (4) includes at least a portion of oblong shape.

3. A device (1) according to claim 1, characterized in that the hole (4) is of oblong shape.

4. A device (1) according to claim 2, characterized in that the hole (4) is of oblong shape.

5. A device (1) according to claim 1, characterized in that said shank (6) is threaded.

6. A device (1) according to claim 2, characterized in that said shank (6) is threaded.

7. A device (1) according to claim 3, characterized in that said shank (6) is threaded.

8. A device (1) according to claim 1, characterized in that the abutment (9) forms a bearing surface for the head (7), said bearing surface extending substantially in a bearing plane (14).

9. A device (1) according to claim 8, characterized in that the assembly part (3) includes an internal face (3C) for placing the support (2), the bearing plane (14) being substantially inclined relative to said internal face (3C).

10. A device (1) according to claim 1, characterized in that the hole (4) includes firstly a bottom stage (10) for placing the support (2) and including a bottom orifice (10A) presenting a section constriction (10B) to form said abutment (9), and secondly a top stage (11) having at least one internally threaded top orifice (11A) for enabling the lock screw (8A, 8B) to be screwed therein.

11. A device (1) according to claim 10, characterized in that the bottom orifice (10A) is oblong in shape extending in a longitudinal direction (Y-Y′), the head (7) coming to bear against the section constriction (10B) and being capable of sliding within the bottom orifice (10A) along the direction (Y-Y′).

12. A device (1) according to claim 10, characterized in that the top stage (10) comprises a plurality of internally threaded top orifices (110A, 111A, 112A) positioned side by side to form a single resulting top orifice (11A) while enabling the lock screw (8A, 8B) to be screwed into each of said internally threaded top orifices (110A, 111A, 112A).

13. A device (1) according to claim 11, characterized in that the top stage (10) comprises a plurality of internally threaded top orifices (110A, 111A, 112A) positioned side by side to form a single resulting top orifice (11A) while enabling the lock screw (8A, 8B) to be screwed into each of said internally threaded top orifices (110A, 111A, 112A).

14. A device (1) according to any one of claim 1, characterized in that the support (2) is a bony support, and the assembly part (3) is a surgical instrument or implant.

15. A device (1) according to claim 14, characterized in that the assembly part (3) is an osteosynthesis plate, and in particular an epiphyseal plate or a shaft plate.

16. An assembly part (3) for a device (1) in accordance with claim 1.

17. A method of securing a device (1) to a support (2), said device (1) comprising firstly an assembly part (3) provided with at least one hole (4) defining a hole section (4), and secondly a fastener member (5) for fastening the assembly part (3) to the support (2), said fastener member (5) comprising a shank (6) together with a head (7) surmounting said shank (6) and designed to be received within the hole (4), said method comprising a step of positioning the head (7) in the hole (4), an anchoring step during which the shank (6) is anchored in the support (2), and a step of securing the head (7) with the assembly part (3) with the help of at least one lock screw (8A, 8B), the method being characterized in that the hole (4) is shaped to form an abutment (9) for the head (7), said hole (4) also being shaped and dimensioned so that the head (7), when bearing against the abutment (9), can be moved within the hole (4) in translation substantially in the plane of the section of the hole (4), the hole (4) including at least one internal thread (4A, 4B, 4C) in which the lock screw (8A, 8B) is designed to be screwed, said step of securing the head (7) with the assembly part (3) comprising screwing the lock screw (8A, 8B) into said internal thread (4A, 4B, 4C) so that said lock screw (8A, 8B) bears against the head (7) and thus presses it against the abutment (9).

18. A method according to claim 17, characterized in that it includes a step of moving the head (7) relative to the hole (4), within which the head (7) is moved relative to the hole (4) in translation substantially in the plane of the section of the hole (4).

19. A method according to claim 18, characterized in that the movement step is performed after the anchoring step, by moving the assembly part (3) relative to the support (2).
